(19) United States
(12) Patent Application Publication
(10) Pub. No.: US 2012/0041026 A1
(43) Pub. Date: Feb. 16, 2012

(54) (R)-4-((4-((4-(TETRAHYDROFURAN-3-YLOXY)BENZO[D]ISOXAZOL-3-YLOXY)METHYL)PIPERIDIN-1-YL)METHYL)TETRAHYDRO-2H-PYRAN-4-OL, A PARTIAL AGONIST OF 5-HT4 RECEPTORS

(75) Inventors: Nobuaki Waizumi, Toyonaka-shi (JP); Hirohide Noguchi, Chita-gun (JP)

(73) Assignee: Pfizer Inc.

(21) Appl. No.: 13/028,368

(22) Filed: Feb. 16, 2011

Related U.S. Application Data
(60) Provisional application No. 61/304,921, filed on Feb. 16, 2010.

(51) Int. Cl. C07D 413/14; A61P 25/22; A61P 25/18; A61P 25/24; A61K 31/454; A61P 25/28

(52) U.S. Cl. 514/321; 546/198

(57) ABSTRACT

(R)-4-((4-((4-(tetrahydrofuran-3-yloxy)benzo[d]isoxazol-3-yloxy)methyl)piperidin-1-yl)methyl)tetrahydro-2H-pyran-4-ol and its use in treating neurodegenerative disorders, is described herein.

This application claims the benefit under 35 U.S.C. §119(e) of U.S. Provisional Patent Application No. 61/304,921, filed on Feb. 16, 2010, the disclosure of which is hereby incorporated by reference in its entirety.

### FIELD OF THE INVENTION

The present invention is directed to (R)-4-((4-((4-(tetrahydrofuran-3-yloxy)benzo[d]isoxazol-3-yloxy)methyl)piperidin-1-yl)methyl)tetrahydro-2H-pyran-4-ol and pharmaceutically acceptable salts thereof. This invention also is directed, in part, to a method for treating a 5-HT₄ mediated disorder in a mammal. Such disorders include acute neurological and psychiatric disorders, stroke, cerebral ischemia, spinal cord trauma, head trauma, perinatal hypoxia, cardiac arrest, hypoglycemic neuronal damage, dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, depression, epilepsy, convulsions, migraine, urinary incontinence, substance tolerance, substance withdrawal, psychosis, schizophrenia, anxiety, mood disorders, trigeminal neuralgia, hearing loss, tinnitus, macular degeneration of the eye, gastroesophageal reflux disease, gastrointestinal disease, gastric motility disorder, non-ulcer dyspepsia, functional dyspepsia, irritable bowel syndrome, constipation, dyspepsia, esophagitis, gastroesophageral disease, nausea, emesis, brain edema, pain, tardive dyskinesia, sleep disorders, attention deficit/hyperactivity disorder, attention deficit disorder, disorders that comprise as a symptom a deficiency in attention and/or cognition, and conduct disorder.

### BACKGROUND OF THE INVENTION

The serotonin 5-HT₄ receptor is a G-protein receptor that is widely distributed throughout the brain, including two brain regions that are critical to cognitive processes; the cortex and hippocampus. The receptors are positively coupled to adenylate cyclase and exert their control on neuronal activity through the cyclic adenosine monophosphate (cAMP) second messenger system. Agonist induced activation of neuronal 5-HT₄ receptors is reported to increase neurotransmitter release by inhibiting neuronal calcium activated and voltage sensitive potassium channels. Inhibition of these channels produces a reduction in the after hyperpolarization and a concomitant increase in neuronal excitability (Eglen et al., Trends Pharmacol Sci 1995; 16:391-398). The neurotransmitter acetylcholine is involved in cognition and memory processes and loss of cholinergic function is believed to be a major cause of the cognitive decline seen with Alzheimer's disease (Francis et al., J Neurol Neurosurg Psychiatry 1999; 66:137-47). Agonist activation of 5-HT₄ receptors, possibly located on the cell bodies or nerve terminals of cholinergic neurons, is reported to enhance acetylcholine (ACh) release in the cortex and the hippocampus (King et al., Trends Pharmacol Sci 2008; 29(9): 482-492; Consolo et al., Neuroreport 1994; 5: 1230-1232; Mohler et al., Neuropharmacology 2007; 53:563-573).

5-HT₄ agonists are also reported to reverse the cognitive deficits induced by pharmacological treatment with anticholinergic drugs (ex atropine and scopolamine) in non-clinical behavioral models (Fontana et al., Neuropharmacology 1997; 36(4/5):689-696; Galeotti et al., J Pharmcol Exp Ther 1998; 286(3):1115-21). Hippocampal theta rhythm is a low frequency oscillating field potential that has been strongly linked to several cognitive, memory and attentional processes in both animals and man (McNaughton et al., Behav Pharmacol 2007; 18 (5/6):329-46; McNaughton et al., Hippocampus 2006; 16 (12):1102-10; Kahana, J Neurosci 2006; 26 (6):1669-72). Acetylcholine is thought to play a major role in the regulation of hippocampal theta rhythms (Vertes et al., Neuroscience 1997; 81(4): 893-926) and administration of acetylcholinesterase inhibitors, such as donepezil, have been shown to increase hippocampal theta rhythm in non-clinical models (Kinney et al., J Pharmacol Exp Ther 1999; 291(1):99-106). As 5-HT₄ agonists have been shown to increase acetylcholine levels in brain, increased theta oscillation may contribute to the cognitive effects observed in preclinical animal models.

In addition to modulating neurotransmitter release, 5-HT₄ agonists may increase levels of soluble amyloid precursor protein alpha (sAPPα). Decreased levels of sAPPα in cerebral spinal fluid (CSF) have been associated with cognitive decline in aged rats (Anderson et al., Neuroscience 1999; 93(4): 1409-1420). Reductions in sAPPα have also been reported in CSF obtained from Alzheimer's patients (Lannfelt et al., Nature Med 1995; 1(8):829-832; Olsson et al., Exp Neurology 2003; 183: 74-80). This may be a consequence of reduced α-secretase activity; the enzyme responsible for sAPPα production (Tyler et al., Biochem Biophys Res Comm 2002; 299: 373-376). Furthermore, in vitro and in vivo studies have reported that activation of 5-HT₄ receptors increases levels of sAPPα (Cachard-Chastel et al., Behav Brain Res 2008; 187:455-461; Cachard-Chastel et al., Brit J Pharmacol 2007; 883:883-892; Mohler et al., Neuropharmacology 2007; 53:563-573) and in some cases decreases the release of Aβ peptides (Cho et al., Exp Neurology 2007; 203:274-278). These results suggest that 5-HT₄ agonists may reduce the production of plaque forming Aβ peptides by diverting amyloid precursor protein away from the amyloidgenic β-secretase pathway to the non-amyloidgenic α-secretase pathway.

Compounds having superior brain penetration are desirable in the treatment of CNS related disorders. Such compounds will freely cross the blood/brain barrier.

A compound having partial agonism of 5-HT₄ may be desirable for treatment of 5-HT₄ mediated disorders, including CNS-related disorders, where it is preferable to reduce or avoid undesirable increases in intestinal motility and other side effects which may result from treatment with 5-HT₄ full agonists.

Co-owned PCT Publication No. WO 06/90224 describes benzisoxazole derivatives having selective 5-HT₄ receptor agonistic activity. These compounds are described as useful for the treatment of gastroesophageal reflux disease, gastrointestinal disease, gastric motility disorder, non-ulcer dyspepsia, functional dyspepsia, irritable bowel syndrome (IBS), constipation, dyspepsia, esophagitis, gastroesophageral disease, nausea, central nervous system disease, Alzheimer's disease, cognitive disorder, emesis, migraine, neurological disease, pain, cardiovascular disorders, cardiac failure, heart arrhythmia, diabetes, and apnea syndrome.

### SUMMARY OF THE INVENTION

The present invention is directed to (R)-4-((4-((4-(tetrahydrofuran-3-yloxy)benzo[d]isoxazol-3-yloxy)methyl)piperidin-1-yl)methyl)tetrahydro-2H-pyran-4-ol, hereinafter referred to as “Compound X,” and having the following structure:

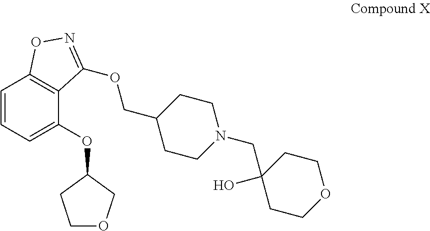

Compound X is a partial agonist of 5-HT₄ receptors which freely crosses the blood brain barrier.

This invention also includes pharmaceutically acceptable salts, hydrates, solvates, isomers, crystalline and non-crystalline forms, isomorphs, and polymorphs of Compound X. This invention also includes all tautomers and stereochemical isomers of these compounds.

This invention also is directed, in part, to a method for treating a 5-HT₄ mediated disorder in a mammal. Such disorders include acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, gastroesophageal reflux disease, gastrointestinal disease, gastric motility disorder, non-ulcer dyspepsia, functional dyspepsia, irritable bowel syndrome, constipation, dyspepsia, esophagitis, gastroesophageral disease, nausea, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, disorders that comprise as a symptom a deficiency in attention and/or cognition, Lewy Body Dementia, and conduct disorder. The method comprises administering Compound X or a pharmaceutically acceptable salt thereof, to the mammal in an amount that is therapeutically effective to treat the condition.

### DETAILED DESCRIPTION OF THE INVENTION

One embodiment of the present invention is Compound X as described above, or a pharmaceutically acceptable salt thereof.

Another embodiment of the present invention is a pharmaceutical composition comprising Compound X, or a pharmaceutically acceptable salt thereof, and a pharmaceutically acceptable carrier.

Another embodiment of the present invention is a method of treating a neurodegenerative disease or disorder, the method comprising administering Compound X, or a pharmaceutically acceptable salt thereof.

Another embodiment of the present invention is a method of treating a neurodegenerative disease or disorder, the method comprising administering Compound X, or a pharmaceutically acceptable salt thereof, wherein the neurodegenerative disease or disorder is dementia, Alzheimer's disease, depression, psychosis, schizophrenia, anxiety, mood disorders, attention deficit/hyperactivity disorder, or attention deficit disorder.

### ABBREVIATIONS AND DEFINITIONS

As used herein the term “Compound X” may be hereinafter referred to as a “compound(s) of the invention.” Such terms are also defined to include all forms of Compound X, including hydrates, solvates, isomers, crystalline and non-crystalline forms, isomorphs, polymorphs, and metabolites thereof.

The following abbreviations are used herein:

| | | | |
|---|---|---|---|
| CD₃OD: | Deuterated methanol | mg: | Milligram |
| CDCl₃: | Deuterated chloroform | MHz: | Megahertz |
| d: | Doublet | min: | Minutes |
| brs: | Broad singlet | mL: | Milliliter |
| g: | Gram | N: | Normal |
| EtOAc: | Ethyl acetate | NMR: | Nuclear magnetic |
| h: | Hour or Hours | | resonance |
| HRMS: | High-resolution mass | ppm: | Parts per million |
| | spectrometry | q: | Quartet |
| J: | Coupling constant | RT: | Room temperature |
| m: | Multiplet | s: | Singlet |
| LRMS: | Low-resolution mass | t: | Triplet |
| | spectrometry | THF: | Tetrahydrofuran |
| M: | Molar | | |

### Tautomeric Forms

The present invention comprises the tautomeric forms of Compound X. Where structural isomers are interconvertible via a low energy barrier, tautomeric isomerism ('tautomerism') can occur. This can take the form of proton tautomerism in Compound X containing, for example, an imino, keto, or oxime group, or so-called valence tautomerism in compounds which contain an aromatic moiety. It follows that a single compound may exhibit more than one type of isomerism. The various ratios of the tautomers in solid and liquid form are dependent on the various substituents on the molecule as well as the particular crystallization technique used to isolate a compound.

### Salts

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining Compound X with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sulfanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, and undecanoate.

Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diethanolamine, glycine, lysine, meglumine, ethanolamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (i.e., dimethyl, diethyl, dibutyl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

### Isotopes

The present invention also includes isotopically labeled compounds, which are identical to Compound X, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the invention can generally be prepared by carrying out the procedures disclosed in the Examples below, by substituting a readily available isotopically labeled reagent for a non-isotopically labeled reagent.

The invention also relates to prodrugs of Compound X. Certain derivatives of Compound X which may have little or no pharmacological activity themselves can, when administered into or onto the body, be converted into Compound X having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series, 1975 (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in Compound X with certain moieties known to those skilled in the art as ‘pro-moieties’ as described, for example, in Design of Prodrugs by H. Bundgaard (Elsevier, 1985).

Some non-limiting examples of prodrugs in accordance with the invention include:
(i) an alcohol functionality which is functionalized into a suitably metabolically labile group (esters, carbonates, carbamates, acetals, ketals, etc.) on Compound X; and
(ii) a primary or secondary amino functionality, or an amide which is functionalized into a suitably metabolically labile group, e.g., a hydrolyzable group (amides, carbamates, ureas, phosphonates, sulfonates, etc.) on Compound X.

Further examples of replacement groups in accordance with the foregoing examples and examples of other prodrug types may be found in the aforementioned references.

### Administration and Dosing

Typically, a compound of the invention is administered in an amount effective to treat a condition as described herein. The compounds of the invention are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg. In another embodiment, the total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than four times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/min during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### Use in the Preparation of a Medicament

In another embodiment, the invention comprises the use of one or more compounds of the invention for the preparation of a medicament for the treatment of the conditions recited herein.

### Pharmaceutical Compositions

For the treatment of the conditions referred to herein, the compound of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, Compound X is ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (i.e., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, Finnin and Morgan, J. Pharm. Sci., 1999, 88, 955-958.

Formulations suitable for topical administration to the eye include, for example, eye drops wherein the compound of this invention is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed-linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

### Co-Administration

The compounds of the present invention can be used, alone or in combination with other therapeutic agents, in the treatment of various conditions or disease states. The compound(s) of the present invention and other therapeutic agent(s) may be may be administered simultaneously (either in the same dosage form or in separate dosage forms) or sequentially. In one embodiment, the other therapeutic agent is dimebon (2,3,4,5-tetrahydro-2,8-dimethyl-5-(2-(6-methyl-3-pyridyl)-ethyl)-1H-pyrid(4,3b)indole). Another exemplary therapeutic agent may be, for example, an NMDA antagonist, acetylcholinesterase (AChE) inhibitor, a PDE 9 inhibitor, or a histamine H3 receptor antagonist.

Examples of NMDA antagonists suitable for co-administration with Compound X include, but are not limited to, 2-Amino-4-[3′-hydroxyphenyl]-4-hydroxybutanoic acid, acamprosate, AM-101 (see http://clinicaltrials.gov/ct2/show/NCT00860808), AZD-6765 (see http://www.clinicaltrials.gov/ct2/show/NCT00491686), budipine, CNS-5161 (3-(2-chloro-5-(methylthio)phenyl)(methyl)(3-(methylthio)phenyl)guanidine), CR-2249 (see Garofalo et al., J. Pharm. Pharmacol., 1996, 48:1290-1297), CR-3394 (see Sarre et al., Eur. J. Pharmacol., 2008, 584:297-305), CR-3991 (see Garofalo et al., Soc. Neurosci. Abstracts 2001, 27:Abs 564.8), dimiracetam, EVT-101 (5-(4-fluoro-3-(difluoromethyl)phenyl)-3-(2-methyl-1H-imidazol-1-yl)methyl)pyridazine), EVT-103 (see http://www.evotec.com/display/articleCategorizedDetail/cms_article_id/11/web site_part_id/4/selected_category_id/6), flupirtine, himantane, huperzine A, indantadol, memantine, mimopezil, NA-1 (see http://clinicaltrials.gov/ct2/show/NCT00728182), neboglamine, neramexane, bis-(7)-tacrine, Neu-120 (see http://clinicaltrials.gov/ct2/show/NCT00607451), Neu-2000 (5-(2,3,5,6-tetrafluoro-4-(trifluoromethyl)benzylamino)-2-hydroxybenzoic acid), NT-13317 (dihydro-1-p-tolyl-1H-pyrrolo[1,2-a]imidazole-2,5(3H,6H)-dione), NVA-011 (see Gonzalez et al., Colloque de la Société des neurosciences (2007), 23 (Abs D.22)), perzinfotel and produgs thereof, radiprodil, ralfinamide, TIK-101 (d-cycloserine), topiramate, or YT-1006 (see http://www.yaupontherapeutics.com/products.html), or a pharmaceutically acceptable salt thereof.

Other examples of NMDA antagonists suitable for co-administration with Compound X include, but are not limited to, the NMDA antagonists disclosed in U.S. Patent Application Publication Nos. US 2007/197594 or US 2009/124600, or in PCT Publication Nos. WO 02/72542, WO 02/80928, WO 03/10159, WO 04/108705, WO 06/10964, WO 06/10965, WO 06/10966, WO 06/10967, WO 06/10969, WO 07/16357, WO 08/137,474, WO 08/138,200, WO 08/91901, WO 09/06437, WO 09/129,181, WO 09/137,843, WO 09/92324, WO 92/15565, WO 97/12870, or WO 98/14427, or a pharmaceutically acceptable salt thereof.

Examples of AChE inhibitors suitable for co-administration with the compounds of the present invention include, but are not limited to, (−)-phenserine, acotiamide, bis-(7)-tacrine, BZYX (see Zhang et al., Eur. J. Pharmacol., 2009, 613:1-9), desoxypeganine, donepezil, EN-101 (see Argov et al., Neurology, 2007, 69: 699-700), galantamine, huperzine A, huprines, INM-176 (see “Drugs under clinical trials in 2005,” Pharma Koreana, 2005, 15: 82-89), itopride, malathion, memogain (see Popa et al., J. Mol. Neurosci., 2006, 30:227-232), memoquin, methanesulfonyl fluoride, metrifonate, mimopezil, NP-61 (see http://www.noscira.com/investigacion.cfm?mS=228&mSS=252), physostigmine, rivastigmine, SP-004 (dimethyl carbamic acid 2,3-bis-di methylcarbamoyloxy-6-(4-ethyl-piperazine-1-carbonyl)-phenyl ester), TA2-PZ5 (see Manetsch et al., J. Am. Chem. Soc., 2004, 126:12809-12818), TA2-PZ6 (see Manetsch et al., supra), tacrine, TZ2-PA5 (see Manetsch et al., supra), TZ2-PA6 (see Bourne et al., Proc. Nat. Acad. Sci., 2004, 101: 1449-1454), or UR-1827 (see Anpeiji et al., Japan. J. Pharmacol., 1999, 79:Suppl I), or a pharmaceutically acceptable salt thereof.

Other examples of AChE inhibitors suitable for co-administration with Compound X include, but are not limited to, the acetylcholinesterase inhibitors disclosed in Chinese Patent Publication No. CN 101440061, European Patent Publication No. EP 1891954, U.S. Patent Application Publication No. US 2009/149444, or in PCT Publication Nos. WO 05/05413, WO 06/39767, WO 07/107,846, WO 07/122,274, WO 08/74816, WO 09/104,990, WO 09/36235, WO 96/26196, WO 97/37992, WO 97/38993, WO 98/00412, WO 98/05292, or WO 98/06697, or a pharmaceutically acceptable salt thereof.

Examples of PDE 9 inhibitors suitable for co-administration with Compound X include, but are not limited to, PF-4447943 (see http://clinicaltrials.gov/ct2/show/NCT00930059), or a pharmaceutically acceptable salt thereof.

Examples of histamine H3 receptor antagonists suitable for co-administration with Compound X include, but are not limited to, APD-916 (see Covel et al., J Med Chem, 2009, 52:5603-5611), CEP-26401 (see Le et al., Soc Neurosci Annual Meeting, 2008, 38: Abs 824.13), ciproxifan, 11C-MK-8278 (see Sanabria-Bohorquez et al., Abs Soc Nuclear Med Ann Meeting, 2009, Abs 1212), ABT-288 (see Esbenshade et al., Soc Neurosci Ann Meeting 2009, Abs 715.23/C13), HPP-404 ((7-chloro-2-(4-cyclopropylpiperazin-1-yl)quinolin-5-yl)(cyclopropyl)methanone), SAR-110894 (see Guillot et al., Soc Neurosci Ann Meeting, 2008, 38th: (Abs 160.21), GSK-835726 (see Ford et al., Allergy, 2009, 64:Suppl 90 (69)), GSK-1004723 (see Clark et al., Allergy, 2009, 64:Suppl 90 (129), GSK-239512 (see http://clinicaltrials.gov/ct2/results?term=NCT01009255), JNJ-17216498 (see http://clinicaltrials.gov/ct2/results?term=NCT00424931), PF-3654746 (see http://clinicaltrials.gov/ct2/results?term=NCT01006122), or pitolisant, or a pharmaceutically acceptable salt thereof.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### Kits

The present invention further comprises kits that are suitable for use in performing the methods of treatment described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

### Intermediates

In another embodiment, the invention relates to the novel intermediates useful for preparing the compounds of the invention.

### Experimental Procedures and Working Examples

Compound X may be prepared by the methods described below, together with synthetic methods known in the art of organic chemistry, or modifications and derivatizations that are familiar to those of ordinary skill in the art. The starting materials used herein are commercially available or may be prepared by routine methods known in the art (such as those methods disclosed in standard reference books such as the Compendium of Organic Synthetic Methods, Vol. I-XII (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in Greene, Protective Groups in Organic Chemistry, John Wiley & Sons, 1981; Greene and Wuts, Protective Groups in Organic Chemistry, John Wiley & Sons, 1991; and Greene and Wuts, Protective Groups in Organic Chemistry, John Wiley & Sons, 1999, which are hereby incorporated by reference.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They are not to constrain the scope of the invention in any way.

The following illustrate the synthesis of various compounds of the present invention. Additional compounds within the scope of this invention may be prepared using the methods illustrated in these Examples, either alone or in combination with techniques generally known in the art.

Experiments were generally carried out under inert atmosphere (nitrogen or argon), particularly in cases where oxygen- or moisture-sensitive reagents or intermediates were employed. Commercial solvents and reagents were generally used without further purification unless indicated otherwise, including anhydrous solvents where appropriate (generally Sure-Seal™ products from the Aldrich Chemical Company, Milwaukee, Wis.). Chemical shifts for nuclear magnetic resonance (NMR) data are expressed in parts per million (ppm, δ) referenced to residual peaks from the deuterated solvents employed.

For syntheses referencing procedures in other Examples, reaction conditions (length of reaction and temperature) may vary. In general, reactions were followed by thin layer chromatography or mass spectrometry, and subjected to work-up when appropriate. Purifications may vary between experiments: in general, solvents and the solvent ratios used for eluants/gradients were chosen to provide appropriate R_{f}s or retention times.

### Example 1

### Synthesis of (R)-4-((4-((4-(tetrahydrofuran-3-yloxy)benzoklisoxazol-3-yloxy)methyl)piperidin-1-yl)methyl)tetrahydro-2H-pyran-4-ol

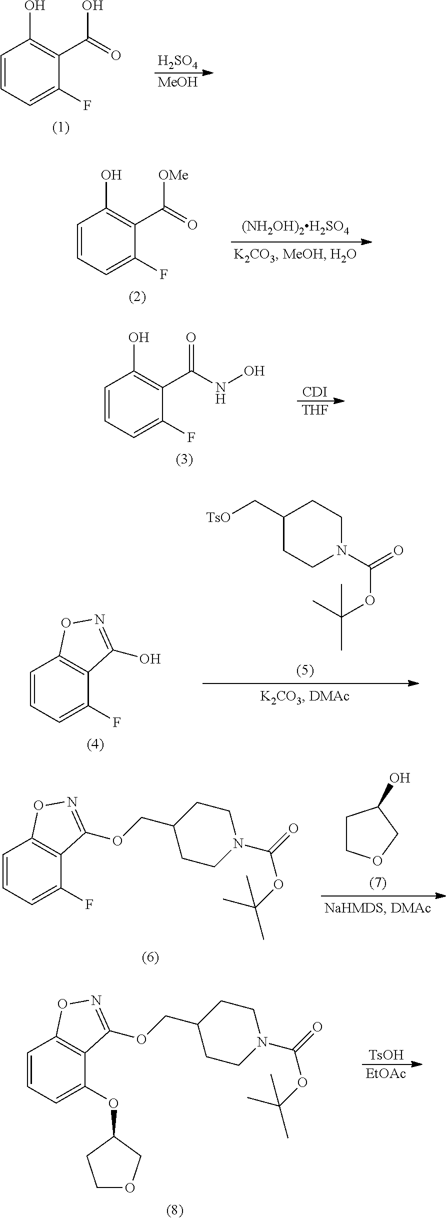

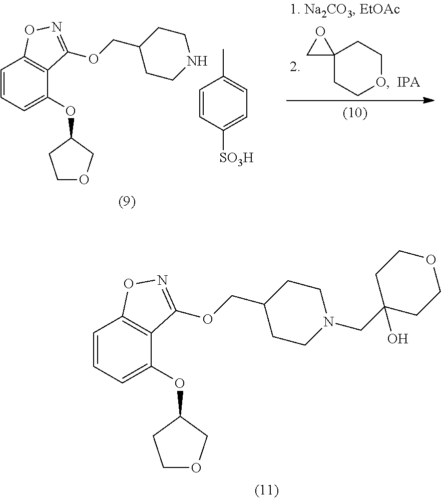

Methyl 2-fluoro-6-hydroxybenzoate (2): To a 20 L jacketed reactor were charged 2-fluoro-6-hydroxybenzoic acid (Oakwood Products; 0.972 kg, 6.31 mol), methanol (7.60 L) and sulfuric acid (0.710 kg, 7.24 mol, 1.15 eq). The jacket temperature was heated to 60° C. and the reaction mixture was stirred for 45 h. The reaction mixture was concentrated under vacuum and approximately 7.5 L of methanol distillates were collected. The resulting thin oil was cooled to 20° C. Water (7.60 L) and ethyl acetate (7.60 L) were charged to the reactor, and the product extracted into the organic layer. The EtOAc solution was washed with a solution of sodium bicarbonate (1.52 Kg) in water (6.92 L) followed by a brine solution of sodium chloride (1.74 kg) in water (4.08 L). The resulting EtOAc solution was concentrated to dryness. A light orange oil was isolated; the oil slowly crystallized upon standing to give the title compound (2) (0.952 Kg, 5.60 mol, 89% yield). ¹H NMR (400 MHz, CDCl₃) δ ppm 3.97 (s, 3H), 6.59 (ddd, J=10.9, 8.2, 1.2, 1H), 6.76 (dt, J=8.2, 1.1, 1H), 7.35 (td, J=8.6, 6.3, 1H), 11.24 (s, 1H); ¹³C NMR (400 MHz, CDCl₃) δ ppm 52.65, 102.56 (d, J=13), 106.90 (d, J=23), 113.31 (d, J=3.1), 135.34 (d, J=11.5), 161.02, 163.31 (d, J=62.2), 169.87 (d, 3.8); MS 171.045 (m+1). 2-Fluoro-N,6-dihydroxybenzamide (3): To a 50 L reactor was charged water (4.47 L) and hydroxylamine sulfate (6.430 kg, 39.17 mol), the mixture was stirred at 25° C. A solution of potassium carbonate (3.87 Kg, 27.98 mol) in water (5.05 L) was slowly added to the reaction mixture to form a thick white mixture that was stirred at 20° C. A solution of methyl 2-fluoro-6-hydroxybenzoate (2) (0.952 Kg, 5.60 mol) in methanol (9.52 L) was slowly added to the reactor resulting in mild off gassing. The reaction mixture was then heated to 35° C. and stirred for 20 h. The reaction mixture was cooled to 15° C. and stirred for 1 h. The mixture was filtered to remove inorganic material. The reactor was rinsed with methanol (2.86 L) and the tank rinse was used to wash the inorganic cake.

Analysis of the cake indicated that it contained product. To a 20 L reactor was charged methanol (10 L) and the inorganic cake and the mixture was stirred at 25° C. for 30 min. The mixture was filtered and the cake washed with methanol (3 L).

The combined filtrates were charged back into the reactor and concentrated under vacuum with the jacket temperature set at 40° C. until approximately 10 L remained. The mixture was held at 25° C. and conc. HCl (5.51 L) was added. The reactor was cooled to 15° C. and stirred for 2 h. The white slurry was filtered and the resulting product cake was washed with water (4.76 L), blown dry with nitrogen and then dried in a vacuum oven at 40° C. for 12 h. The desired product (3) (747 g, 4.36 mol), was isolated in 78% yield. ¹H NMR (400 MHz, CD₃OD) δ ppm 4.91 (s, 3H), 6.63 (ddd, J=10.9, 8.5, 0.8, 1H), 6.72 (dt, J=8.2, 0.8, 1H), 7.31 (td, J=8.2, 6.6, 1H); MS 172.040 (m+1).

4-Fluorobenzo[d]isoxazol-3-ol (4): To a 20 L jacketed reactor were charged tetrahydrofuran (2.23 L) and 1,1′-carbonyldiimidazole (0.910 Kg, 5.64 mol). The resulting mixture was stirred at 20° C. Then a solution of 2-fluoro-N,6-dihydroxybenzamide (3) (744 g, 4.34 mol) in tetrahydrofuran (4.45 L) was slowly charged to the reactor maintaining the temperature below 30° C. and stirred at 25° C. for 30 min during which some off gassing was observed. The reaction mixture was heated to 60° C. over 30 min and stirred for 6 h. The reactor was cooled to 20° C. followed by the addition of 1N aqueous hydrogen chloride (7.48 L) over 15 min to adjust the pH to 1. The jacket temperature was set to 35° C. and the reaction mixture concentrated under vacuum to remove approximately 6.68 L of THF. The reactor was cooled to 15° C. and stirred for 1 h. The resulting white slurry was filtered, the cake was washed with water (3.71 L) and dried in a vacuum oven at 40° C. for 12 h. The desired product, (4) (597 g, 3.90 mol), was isolated in 90% yield. ¹H NMR (400 MHz, CD₃OD) δ ppm 4.93 (b, 1H), 6.95 (dd, J=10.1, 8.6, 1H), (d, J=8.6, 1H), 7.52-7.57 (m, 1H); LRMS 154.029 (m+1).

Tert-butyl 4-(tosyloxymethyl)piperidine-1-carboxylate (5): To a 20 L jacketed reactor were charged dichloromethane (8 L), N-boc-4-piperidine methanol (0.982 Kg, 4.56 mol) and p-toluenesulfonyl chloride (0.970 Kg, 5.09 mol) and the resulting mixture was stirred at 20° C. for 5 min. Triethylamine (0.94 Kg, 9.29 mol) was added to the reactor via an addition funnel and the resulting deep red solution was stirred at 25° C. for 16 h. A solution of sodium carbonate (0.96 Kg, 9.06 mol) in water (7.04 L) was charged to the reaction mixture and stirred for 1 h at 20° C. The phases were split and the organic layer washed with brine (6 L) and concentrated at 40° C. to a low stir volume. Dimethylacetamide (2 L) was charged to the reactor and concentration continued under full vacuum at 40° C. for 1 h. The solution of tert-butyl 4-(tosyloxymethyl)piperidine-1-carboxylate (5) in dimethyl acetamide was held for further processing. Yield was assumed to be 100% with approximately 90% potency. A sample was pulled and concentrated to dryness for purity analysis. ¹H NMR (400 MHz, CDCl₃) δ ppm 1.02-1.12 (m, 2H), 1.14 (s, 9H), 1.59-1.64 (m, 2H), 1.75-1.87 (m, 1H), 2.43 (s, 3H), 2.55-2.75 (m, 2H), 3.83 (d, J=6.7, 2H), 3.95-4.20 (b, 2H), 7.33 (d, 8.6, 2H), 7.76 (d, 8.2, 2H); ¹³C NMR (400 MHz, CDCl₃) δ ppm 21.64, 28.15, 28.39, 35.74, 73.97, 79.50, 126.99, 127.84, 129.86, 132.84, 144.84, 154.63; LRMS 739.329 (2 m+1).

Tert-butyl ((4-fluorobenzo[d]isoxazol-3-yloxy)methyl)piperidine-1-carboxylate (6): To a 20 L jacketed reactor were charged dimethylacetamide (4.28 L), tert-butyl 4-(tosyloxymethyl)piperidine-1-carboxylate (5) (1.68 Kg, 4.56 mol), 4-fluorobenzo[d]isoxazol-3-ol (4) (540 g, 3.51 mol), and potassium carbonate (960 g, 6.98 mol) resulting in a thick beige slurry. The reaction mixture was heated to 50° C. and stirred for 20 h and then cooled to 20° C., followed by the addition of water (7.5 L) and ethyl acetate (5.37 L). After mixing for 15 min, the phases were settled and split. The organic layer was washed with water (5.37 L), sending the aqueous wash to waste. The organic mixture was distilled under vacuum with a maximum jacket temperature of 40° C. until approximately 5 L remained in the reactor. Methanol (2.68 L) was added and the resulting solution concentrated under vacuum to about 3 L of a yellow oil. Methanol (2.68 L) was charged to the reactor and the resulting solution was stirred at 25° C. for 15 min. Water (0.54 L) was added over 15 min resulting in a white slurry. The mixture was cooled to 15° C., stirred for 1 h and then filtered. The filter cake was washed with a solution of water (0.54 L) in methanol (2.14 L), then air dried for 30 min, transferred to a vacuum oven and dried at 40° C. for 12 h. The desired product, (6) (746 g, 2.13 mol), was isolated in 61% yield. ¹H NMR (400 MHz, CDCl₃) δ ppm 1.23-1.37 (m, 2H), 1.45 (s, 9H), 1.78-1.88 (m, 2H), 2.04-2.17 (m, 1H), 2.67-2.83 (m, 2H), 4.02-4.26 (m, 2H), 4.28 (d, 6.6, 2H), 6.89 (dd, J=8.6, 7.5, 1H), 7.21 (d, J=9, 1H), (td, 8.6, 4.9); LRMS 351.171 (m+1).

(R)-Tert-butyl ((4-(tetrahydrofuran-3-yloxy)benzo[d]isoxazol-3-yloxy)methyl)piperidine-1-carboxylate (8): To a 20 L glass reactor with the jacket set to 20° C. were charged (R)-tetrahydrofuran-3-ol (7) (297 g, 3.37 mol) and dimethylacetamide (5.1 L). 2.0 M sodium bis(trimethylsilyl)amide in THF (1.37 L, 2.74 mol) was slowly added via an addition funnel while maintaining a pot temperature less than 30° C. The resulting orange/red solution was stirred at 25° C. for 30 min. Then, tert-butyl 44(4-fluorobenzo[d]isoxazol-3-yloxy)methyl)piperidine-1-carboxylate (6) (640.15 g, 1.83 mol) was charged and the reaction mixture was stirred at 25° C. for 16 h. The reaction mixture was cooled to 20° C. and water (6.4 L) was slowly added over 45 min maintaining a pot temperature of less than 35° C. Ethyl acetate (6 L) was added and the biphasic mixture was stirred for 15 min and then separated. The aqueous layer was back extracted with additional ethyl acetate (4 L). The combined organics were then washed with water (5 L) and a 20% brine solution (5 L). The organic mixture was concentrated under vacuum with the jacket temperature set to 40° C. to approximately 3 L and held for further processing. Quantitative yield of the desired product, (8) (0.76 Kg, 1.82 mol), in ethyl acetate was assumed. A sample was pulled and concentrated to dryness for purity analysis. ¹H NMR (400 MHz, CDCl₃) δ ppm 1.25-1.38 (m, 2H), 1.44 (s, 9H), 1.76-1.84 (m, 2H), 1.89-1.97 (b, 1H), 1.99-2.12 (m, 1H), 2.14-2.28 (m, 2H), 2.63-2.84 (m, 2H), 3.90-4.21 (m, 6H), 4.24 (d, J=6.3, 2H), 5.00-5.05 (m, 1H), 6.48 (d, J=8.2, 1H), 6.98 (d, J=8.6, 1H), 7.37 (t, J=8.2, 1H); LRMS 419.216 (m+1).

(R)-3-(Piperidin-4-ylmethoxy)-4-(tetrahydrofuran-3-yloxy)benzo[d]isoxazole 4-methylbenzenesulfonate (9): To a 20 L jacketed reactor charged ethyl acetate (6.1 L), (R)-tert-butyl 44(4-(tetrahydrofuran-3-yloxy)benzo[d]isoxazol-3-yloxy)methyl)piperidine-1-carboxylate (8) (0.76 kg, 1.82 mol) and p-toluenesulfonic acid monohydrate (0.413 kg, 2.17 mol) and stirred at 20° C. for 30 min. The reactor jacket was heated from 20 to 65° C. over 1 h and then held at 65° C. for 16 h. The reactor was cooled to 15° C. over 1 h and granulated for 2 h. The resulting slurry was filtered, the cake was washed with EtOAc (3 L) and then air dried on the filter for 30 min. The cake was transferred to a vacuum oven and dried at 40° C. for 12 h. The desired product, (9) (854 g, 1.74 mol), was isolated in 96% yield (two steps). ¹H NMR (400 MHz, CD₃OD) δ ppm 1.54-1.67 (m, 2H), 2.04-2.18 (m, 3H), 2.19-2.36 (m, 2H), 2.33 (s, 3H), 3.01-3.12 (m, 2H), 3.41-3.50 (m, 2H), 3.86-4.01 (m, 4H), 4.26 (d, J=6.3, 2H), 4.90 (s, 2H), 5.14-5.19 (m, 1H), 6.72 (d, J=8.2, 1H), 7.02 (d, J=8.6, 1H), 7.21 (d, J=7.8, 2H), 7.48 (t, J=8.6, 1H), 7.70 (d, J=8.2, 2H); LRMS 319.165 (m+1).

(R)-4((4-((4-(Tetrahydrofuran-3-yloxy)benzo[d]isoxazol-3-yloxy)methyl)piperidin-1-yl)methyl)tetrahydro-2H-pyran-4-ol (11): To a 20 L jacketed reactor were charged water (7.5 L) and sodium carbonate (0.98 kg); the mixture was stirred at 20° C. until all solids had dissolved. Then (R)-3-(piperidin-4-ylmethoxy)-4-(tetrahydrofuran-3-yloxy)benzo[d]isoxazole 4-methylbenzenesulfonate (9) (750 g, 1.53 mol) and ethyl acetate (6.0 L) were added to the reactor and stirred at 20° C. for 30 min. The phases were split and the lower aqueous layer was back extracted twice with ethyl acetate (6.0 L and then 3.75 L). The organic layers were combined in the 20 L reactor and washed twice with brine (3.0 L). The ethyl acetate solution was concentrated to under vacuum at 45° C. to a low stir volume. Isopropyl alcohol (3.75 L) was added and concentration continued until 2 L remained in the reactor. Additional isopropyl alcohol (2.75 L) was added and the mixture cooled to 25° C. To the reactor was charged 1,6-dioxaspiro[2.5]octane (10) (260 g, 2.29 mol) and the resulting solution heated to 50° C. and stirred for 16 h. The reaction mixture was cooled to 30° C. and water (15 L) was added over 60 min. Product crystallized from solution and the resulting slurry was cooled to 15° C. over 1 h and then granulated for 4 h. The product was filtered and washed with water (3.75 L). The cake was blown dry with nitrogen for 30 min and then transferred to a vacuum oven and dried at 40° C. for 12 h. The desired product, (11) (588 g, 1.36 mol), was isolated in 89% yield. ¹H NMR (400 MHz, CDCl₃) δ ppm 1.41-1.63 (m, 6H), 1.71-1.81 (m, 2H), 1.81-1.94 (m, 1H), 2.17-2.26 (m, 2H), 2.33 (s, 2H), 2.4 (td, J=11.7, 2.3, 2H), 2.92 (d, J=11.8, 2H), 3.46 (s, 1H), 3.71-3.84 (m, 4H), 3.91-4.10 (m, 4H), 4.24 (d, J=5.9, 2H), 5.03-5.08 (m, 1H), 6.50 (d, J=8.2, 1H), 7.00 (d, J=8.2, 1H), 7.38 (t, J=8.2, 1H); ¹³C NMR (400 MHz, CDCl₃) δ ppm 29.11, 33.10, 35.20, 36.92, 36.96, 56.15, 63.93, 67.14, 67.46, 68.27, 72.94, 74.06, 78.37, 103.17, 105.15, 131.71, 152.71, 166.02, 166.28; LRMS 433.232 (m+1).

### Example 2

### Synthesis of (R)-4-((4-((4-(tetrahydrofuran-3-yloxy)benzo[d]isoxazol-3-yloxy)methyl)piperidin-1-yl)methyl)tetrahydro-2H-pyran-4-ol

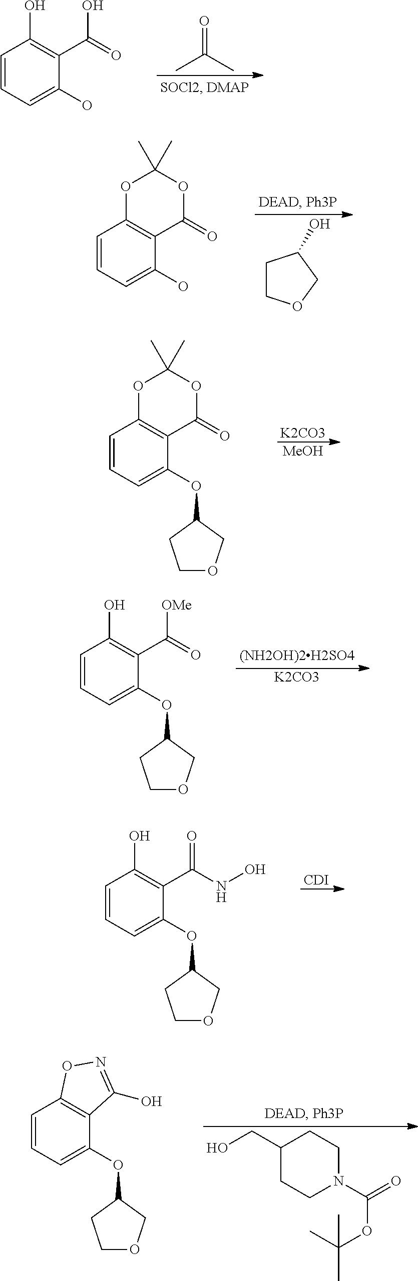

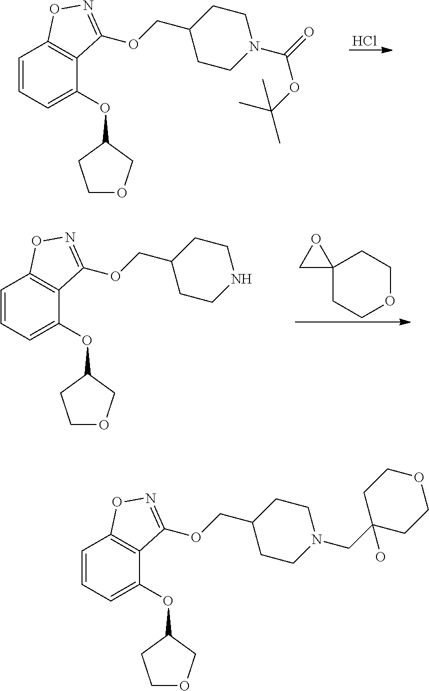

5-Hydroxy-2,2-dimethyl-benzo[1,3]dioxin-4-one: Thionyl chloride (83.8 g, 0.71 mol) was slowly added to a solution of 2,6-dihydroxy-benzoic acid (77 g, 0.5 mol), acetone (37.7 g, 0.65 mol) and DMAP (3.1 g, 0.025 mol) in dimethoxyethane (375 mL). The mixture was stirred at RT for 7 h. The residue obtained after concentration under reduced pressure was dissolved in ethyl acetate and washed with water and aqueous saturated sodium bicarbonate solution. The organic layer was dried (Na₂SO₄) and concentrated to afford 79 g desired product as a red solid (81% yield). ¹H NMR (400 MHz, CDCl₃) δ ppm 1.68 (s, 6H), 6.37 (dd, J=8, 0.8, 1H) 6.56 (dd, J=8, 0.8, 1H), 7.34 (t, J=8, 1H), 10.27 (brs, 1H).

2,2-Dimethyl-5-[(R)-(tetrahydro-furan-3-yl)oxy]-benzo[1,3]dioxin-4-one: Diethyl azodicarboxylate (130.5 g, 0.75 mol) was added in a dropwise fashion to a mixture of 5-hydroxy-2,2-dimethyl-benzo[1,3]dioxin-4-one (100 g, 0.51 mol), triphenylphosphine (196.5 g, 0.75 mol), and (S)-tetrahydro-furan-3-ol (44 g, 0.5 mol) in 600 mL of anhydrous THF. The resulting mixture was stirred at RT for 18 h. The solvent was removed under reduced pressure and the crude material was purified on a silica gel flash column, eluting with petroleum ether/ethyl acetate (15:1→3:1). 86 g (65% yield) of product was isolated as a colorless oil. ¹H NMR (400 MHz, CDCl₃) δ ppm 1.67 (s, 6H), 2.30 (m, 2H), 4.2 (m, 4H) 4.97 (m, 1H), 6.49 (d, J=8.4, 1H) 6.51 (d, J=8.4, 1H), 7.39 (t, J=8.4, 1H).

2-Hydroxy-6-[(R)-(tetrahydro-furan-3-yl)oxy]-benzoic acid methyl ester: Potassium carbonate (134.8 g, 0.98 mol) was added to a solution of 2,2-dimethyl-5-[(R)-(tetrahydro-furan-3-yl)oxy]-benzo[1,3]dioxin-4-one (86 g, 0.33 mol) in 1 L methanol. The mixture was stirred at RT for 2 h, then concentrated in vacuo. The residue was dissolved in ethyl acetate and washed with aqueous ammonium chloride solution. The organic layer was dried (Na₂SO₄) and concentrated to afford 72 g of the product as a yellow solid (92% yield). ¹H NMR (400 MHz, CDCl₃) δ ppm 2.20 (m, 2H), 3.99 (s, 3H), 4.80 (m, 4H).

4.94 (m, 1H), 6.31 (dd, J=8.4, 0.8, 1H), 6.59 (dd, J=8.4, 0.8, 1H), 7.30 (t, J=8.4, 1H).

2,N-Dihydroxy-6-[(R)-(tetrahydro-furan-3-yl)oxy]-benzamide: Potassium carbonate (121 g. 0.867 mmol) was added portionwise to a solution of hydroxylamine sulfate (120 g, 0.732 mol) in 360 mL of water at 0° C. After stirring for 30 min, sodium sulfite (3.74 g, 0.029 mol) and a solution of 2-hydroxy-6-[(R)-(tetrahydro-furan-3-yl)oxy]-benzoic acid methyl ester (35 g, 0.146 mol) in 360 mL of methanol were added and the mixture was stirred at 50° C. for 30 h. Methanol was removed from the cooled reaction mixture under reduced pressure and the resulting aqueous layer was acidified with 2N HCl. The aqueous layer was extracted with ethyl acetate and the organic layer was dried (Na₂SO₄) and concentrated to afford 25 g (76% yield) of the product as a yellow solid. ¹H NMR (400 MHz, CDCl₃) δ ppm 2.00 (m, 1H), 2.15 (m, 1H), 3.80 (m, 4H), 5.05 (m, 1H), 6.48 (d, J=8, 1H), 6.49 (d, J=8, 1H), 7.19 (t, J=8, 1H), 10.41 (brs, 1H), 11.49 (brs, 1H); LRMS m/z 239 (m+1).

4-[(R)-(Tetrahydro-furan-3-yl)oxy]-benzo[d]isoxazol-3-ol: A solution of 2,N-dihydroxy-6-[(R)-(tetrahydro-furan-3-yl)oxy]-benzamide (25 g, 0.105 mol) in 250 mL of THF was heated to 50° C. Carbonyl diimidazole was added portionwise and the resulting mixture was stirred at 50° C. for 14 h. After cooling to RT, 100 mL of 2N HCl was added and the aqueous layer was extracted with ethyl acetate. The combined organic layers were then extracted three times with 10% aqueous potassium carbonate. The potassium carbonate aqueous extracts were washed with ethyl acetate and then acidified to pH 2-3 with 2N HCl. The acidified aqueous layer was extracted with ethyl acetate. The ethyl acetate extracts were washed with brine, dried (Na₂SO₄) and concentrated to afford 20 g of product as a yellow solid (43% yield). ¹H NMR (400 MHz, CDCl₃) δ ppm 2.20 (m, 2H), 3.89 (m, 1H), 4.01 (m, 3H), 5.05 (m, 1H), 6.48 (d, J=7.6, 1H). 6.92 (d, J=7.6, 1H), 7.37 (t, J=7.6, 1H); LRMS m/z 222 (m+1).

4-{4-[(R)-(Tetrahydro-furan-3-yl)oxy]-benzo[d]isoxazol-3-yloxymethyl}-piperidine-1-carboxylic acid tert-butyl ester: Diethyl azodicarboxylate (15.6 g, 0.09 mol) was added to a mixture of 4-[(R)-(tetrahydro-furan-3-yl)oxy]-benzo[d]isoxazol-3-ol (10 g, 0.045 mol), 4-hydroxymethyl-piperidine-1-carboxylic acid tert-butyl ester (11.6 g, 0.054 mol) and triphenylphosphine (23.5 g, 0.09 mol) in 300 mL THF. After the addition was complete the mixture was heated at reflux for 18 h. After concentration in vacuo, the crude product was purified on a silica gel flash column, eluting with petroleum ether/ethyl acetate (15:1→5:1) to afford 22 g of the product as an oil (51% yield). ¹H NMR (400 MHz, CDCl₃) δ ppm 1.25 (m, 2H), 1.39 (s, 9H), 1.76 (m, 2H), 1.99 (m, 1H). 2.15 (m, 2H), 2.70 (bt, J=11.6, 2H), 3.95 (m, 4H). 4.13 (m, 2H). 4.34 (d J=6.4, 2H), 4.98 (m, 1H), 6.43 (d, J=8, 1H), 6.93 (d, J=8, 1H), 7.31 (t, J=8, 1H).

3-(Piperidin-4-ylmethoxy)-4-[(R)-(tetrahydro-furan-3-yl)oxy]-benzo[d]isoxazole: A 0° C. solution of 4-{4-[(R)-(tetrahydro-furan-3-yl)oxy]-benzo[d]isoxazol-3-yloxymethyl}-piperidine-1-carboxylic acid tert-butyl ester in 500 mL ether was treated with a saturated solution of HCl (g) in 200 mL ether. After addition was complete, the mixture was warmed to RT and stirred for 16 h. The reaction mixture was filtered. The white solid was washed with ethyl acetate followed by ether and dried to yield 15 g (81% yield) of the desired product as a white solid. ¹H NMR (400 MHz, CD₃OD) δ ppm 1.51-1.69 (m, 2 H) 2.04-2.19 (m, 3H) 2.22-2.37 (m, 2H) 2.99-3.14 (m, 2H) 3.40-3.51 (m, 2H) 3.85-4.02 (m, 4H) 4.25-4.31 (m, 2H) 5.17 (td, J=3.71, 1.56 Hz, 1 H) 6.72 (d, J=8.00 Hz, 1H) 7.01 (d, J=8.59 Hz, 1H) 7.47 (t, J=8.20 Hz, 1H); LRMS m/z 319 (m+1).

4-(4-(4-[(R)-(Tetrahydro-furan-3-yl)oxypbenzo[d]isoxazol-3-yloxymethyl]-piperidin-1-ylmethyl)-tetrahydro-pyran-4-ol: 1,6-Dioxa-spiro[2.5]octane (Focus Synthesis; 9.7 g, 0.084 mol) and triethylamine (8.6 g, 0.084 mol) were added to a solution of 3-(piperidin-4-ylmethoxy)-4-[(R)-(tetrahydro-furan-3-yl)oxy]-benzo[d]isoxazole (15 g, 0.042 mol) in 200 mL methanol. The resulting solution was heated at reflux for 18 h. The cooled mixture was concentrated and ethyl acetate and water were added to the residue. The layers were separated and the organic extracts were washed with brine, dried (Na₂SO₄) and concentrated to provide 17 g crude product as a yellow oil. The crude material was purified by prep HPLC to afford 10 g of the desired product as a white solid. (50% yield). ¹H NMR (400 MHz, CDCl₃) δ ppm 1.41-1.63 (m, 6H), 1.71-1.81 (m, 2H), 1.81-1.94 (m, 1H), 2.17-2.26 (m, 2H), 2.33 (s, 2H), 2.4 (td, J=11.7, 2.3, 2H), 2.92 (d, J=11.8, 2H), 3.46 (s, 1H), 3.71-3.84 (m, 4H), 3.91-4.10 (m, 4H), 4.24 (d, J=5.9, 2H), 5.03-5.08 (m, 1H), 6.50 (d, J=8.2, 1H), 7.00 (d, J=8.2, 1H), 7.38 (t, J=8.2, 1H); ¹³C NMR (101 MHz, CDCl₃) δ ppm 29.11, 33.10, 35.20, 36.92, 36.96, 56.15, 63.93, 67.14, 67.46, 68.27, 72.94, 74.06, 78.37, 103.17, 105.15, 131.71, 152.71, 166.02, 166.28.

### Example 3

### Assays

The brain penetration, clearance, effect on general cell health, and intrinsic agonist activity at 5-HT₄ receptors was measured as described below for certain compounds. The compounds assayed included Compound X and the nine compounds, disclosed in International Publication No. WO 06/90224, which displayed the lowest intrinsic activity as 5-HT₄ agonists:

| | Example No. |
|---|---|
| Compound No. | in WO 06/90224 |
| A | Example 3 |
| B | Example 1 |
| C | Example 6 |
| D | Example 9 |
| E | Example 11 |
| F | Example 10 |
| G | Example 51 |
| H | Example 31 |
| J | Example 8 |

Compound X has one or more properties which make it superior to compounds exemplified in WO 06/90224. 5-HT₄ partial agonists having low intrinsic activity may offer the opportunity for treatment of CNS-related disorders with the potential benefit of reducing the gastrointestinal effects which may be inherent with 5-HT₄ full agonist agents. Additionally, superior brain penetration is important for treatment of CNS-related disorders. Optimal chemical matter for such indications will freely cross the blood brain barrier. One skilled in the art would not expect agents containing a carboxylic acid moiety to demonstrate appreciable brain penetration; the data shown in Table 1 for compounds A, B and C confirm this expectation. Good clearance and acceptable expected overall safety profile are also important attributes in a CNS drug. Compound X exhibits lower intrinsic activity than compounds exemplified in WO 06/90224 and additionally differentiates based on at least one property such as brain penetration, clearance, or predicted overall safety profile. Properties of example compounds may be appreciated using known methods or by reference to Table 1. Table 1 compares Compound X to Compounds A-J, which were disclosed in WO 06/90224 and which have low intrinsic activity (Eₘₐₓ<40%), as shown in WO 06/90224 (see “Agonist-Induced cAMP Elevation in Human 5-HT4” at page 33). Compound X and Compounds A-J share the same core structure:

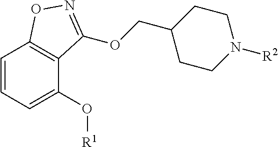

with R¹ and R² groups as shown in Table 1 below.

Brain Penetration Assay: Male Sprague-Dawley rats (n=3/time point) were administered 5 mg/kg of Compounds X, A, B, C, D, E, F, G, H, and J via subcutaneous administration. Blood samples were collected via cardiac puncture after euthanization with CO₂ at 0.5, 1, 2, and 4 h post-dose. Samples were placed in EDTA tubes and kept on ice. Whole brain was collected via decapitation. Brain samples were immediately stored in dry ice. Blood samples were spun down to collect plasma. Plasma and brain samples were stored at −20° C. until analysis. LC/MS/MS was used to measure plasma and brain drug levels. Results are shown in Table 1.

Agonist-induced cAMP elevation in human 5-HT_{4d} transfected HEK293 cells, 96 well format: Human 5-HT_{4(d)} transfected HEK293 cells were established in-house. The cells were grown at 37° C. and 5% CO₂ in DMEM supplemented with 10% FCS, 20 mM HEPES (pH 7.4), 200 μg/mL hygromycin B (Gibco), 100 units/mL penicillin and 100 μg/mL streptomycin. The cells were grown to 60-80% confluence. On the previous day before treatment with compounds, dialyzed FCS (Gibco) was substituted for normal and the cells were incubated overnight. Compounds were prepared in 96-well plates (12.5 μL/well). The cells were harvested with PBS/1 mM EDTA, centrifuged and washed with PBS. At the beginning of the assay, cell pellet was resuspended in DMEM supplemented with 20 mM HEPES, 10 μM pargyline (Sigma) and 1 mM 3-isobutyl-1-methylxanthine (Sigma) at the concentration of 1.6×10⁵ cells/mL and left for 15 min at RT or 37° C. The reaction was initiated by addition of the cells into plates (12.5 μL/well). After incubation for 15 min at RT or 37° C., 1% Triton X-100 was added to stop the reaction (25 μL/well) and the plates were left for 30 min at RT. Homogenous time-resolved fluorescence-based cAMP (Schering) detection was made according to the manufacturer's instruction. ARVOsx multilabel counter (Wallac) was used to measure HTRF (excitation 320 nm, emission 665 nm/620 nm, delay time 50 μs, window time 400 μs). Data was analyzed based on the ratio of fluorescence intensity of each well at 620 nm and 665 nm followed by cAMP quantification using cAMP standard curve. Enhancement of cAMP production elicited by each compound was normalized to the amount of cAMP produced by 1,000 nM serotonin (Sigma). Intrinsic activity is reported in Table 1, below, as % agonist effect.

Agonist-induced cAMP elevation in human 5-HT_{4d} transfected HEK293 cells, 384 well format: Human 5-HT_{4d} transfected HEK293 cells were grown at 37° C. and 5% CO₂ in DMEM (without sodium pyruvate) supplemented with 10% FBS, 20 mM HEPES (pH 7.4) and 200 μg/mL hygromycin B (Gibco). The cells were grown to 60-80% confluence. 24 hours prior to the experiment, the growth media was replaced with Optimem reduced-serum media (Gibco) and the cells were incubated overnight. On the day of the experiment, compounds dissolved in DMSO were diluted in assay buffer containing PBS, 5 uM Hepes, and 500 uM IBMX (final concentrations). The cells were harvested with cell dissociation buffer (Gibco), centrifuged and washed with PBS. The cell pellet was then resuspended in PBS and the cells were counted and diluted appropriately. The reaction was initiated by addition of the cells into 384 well plates containing compounds; the final number of cells used in the assay was 5000 cells per well. After incubation for 30 minutes at 37° C., Cisbio cAMP Dynamic 2 screening kit reagents (cat#62AM4PEB) were added to the plate to stop the reaction. Homogenous time-resolved fluorescence-based cAMP (Schering) detection was determined according to the manufacturer's instruction. A Wallac Envision was used to measure HTRF (excitation 320 nm, emission 665 nm/620 nm, delay time 50 μs, window time 400 μs). Data was analyzed based on the ratio of fluorescence intensity of each well at 620 nm and 665 nm followed by cAMP quantification using a cAMP standard curve. Enhancement of cAMP production elicited by each compound was normalized to the amount of cAMP produced by 1 uM serotonin (Sigma). Intrinsic activity is reported in Table 1, below, as % agonist effect.

Human Liver Microsome Stability Assay: Human liver microsomes (HLMs) are used in a metabolic stability assay to determine the NADPH-dependent in vitro apparent intrinsic metabolic clearance (CL_{int,app}) of a drug (primarily mediated by P450 metabolism). In the HLM assay, test compounds are incubated with HLMs & an NADPH regenerating system in 100 mM potassium phosphate buffer (pH 7.4). The HLMs used in this assay are prepared as a pool from many individual donors. The concentration of HLMs and test compound are 0.71 mg protein/ml and 1 uM, respectively. The reaction is started by the addition of microsomes and buffer to compound. At 0, 5, 10, 20, 30, and 60 minutes, the sample is crashed with ACN/IS (3 times the incubation volume), spun at 4° C. and 3500 rpm for 10 minutes. A matrix and 60 minute w/o NADPH sample are also incubated for 60 minutes serving as negative and positive controls. The matrix sample contains buffer, microsomes, and NADPH (no compound); where as, the 60 minute w/o NADPH sample contains buffer, microsomes, and compound (no NADPH). After centrifugation, the supernatant is removed from the sample, combined with equal parts of water, and stored in the refrigerator until analysis. Drug levels are quantified by mass spectrometry. Clearance is frequently expressed as an extraction ratio (Er) which is calculated as hepatic clearance/hepatic blood flow (range 0-1). The data are shown in Table 1.

THLE assay: The THLE assay is predictive of general cell health and measures cell depletion in a human cell line of hepatic origin. THLE-2 (transformed human liver epithelial) cells were obtained from ATCC(CRL-2706 or CRL-10149) and cultured according to ATCC's recommendation. Media consisted of basal medium (BEGM Bullet Kit, Lonza Cat #CC-3170), supplemented with 10% fetal bovine serum (Sigma Cat #F4135) and 2.5 ng/L hEFG (BD Biosciences Cat #356052) and 700 ng/L phosphoethanolamine (Sigma Cat #p-0503). Cells were cultured in T175 human fibronectin/collagen/bovine serum albumin coated flasks. For each experiment, cells were plated onto 384 well plates (custom order, BD Biosciences Cat #359298) at a cell density of 2.5×10³/well in a total medium volume of 25 μL/well. Plates were incubated for 24 h at 37° C., 5% CO₂.

Compound test plates were prepared using a 10 dose, 2.0 fold dilution scheme with a final assay concentration range from 300-0.058 μM. All compounds were initially solubilized in 100% DMSO. This dosing scheme contained 32 compounds per plate. Stock plates were prepared by aliquoting 1 μL of 100× compound/well (30-0.058 mM). The plates were prepared for dosing by adding 99 μL of cell culture media and mixing. Test compounds were added to cell culture plates by aspirating overnight culture media and replacing with 25 μL/well of media containing test compound using the layout outlined below. The final concentration of DMSO in each well was 1.0%.

Following the 72 h exposure to test compounds, cell viability in each well was determined by measuring the concentration of cellular ATP using the Lonza Vialight™ Plus Cell Proliferation/Cytoxicity Kit (Lonza cat: LT07-121) according to the manufacturer's protocol. The ATP concentration was determined by reading luminescence using a Wallac Envision plate reader (Perkin Elmer, Waltham, Mass., USA). Percent of viable cells relative to no-drug treated controls was determined for each well. Final data output is a calculated IC₅₀ value describing the dose projected to kill 50% of the cells following a 72 h exposure.

TABLE 1

| | | | | Plasma | | | | | |
|---|---|---|---|---|---|---|---|---|---|
| | | | Brain | AUC | Brain: | | | | |
| | | | AUC | (ng*h/ | Plasma | % IA | | | THLE |
| Cmpd | R¹ | R² | (ng*h/g) | mL) | ratio | RT | 37° C. | Er | (μM) |
| X | 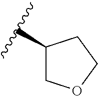 | 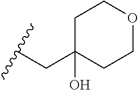 | 1790 | 1760 | 1.02 | 15 | 41^{a} 15^{b} | 0.62 | 300 |
| A | 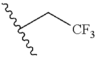 | 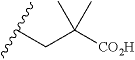 | | | | 35.5 | 98.5^{a} | 0.53 | — |
| B | 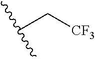 | 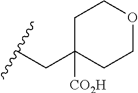 | 160 | 4090 | 0.039 | 56.3 | 103^{a} | TDO | — |
| C | 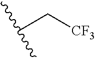 | 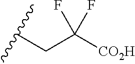 | | | | 30.0 | 88^{a} | — | — |
| D | 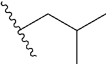 | 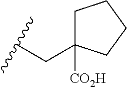 | | | | 36.0 | 95.0^{a} | 0.36 | — |
| E | 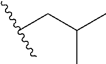 | 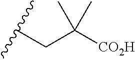 | | | | 29.0 | 97.0^{a} | 0.30 | — |
| F | 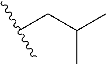 | 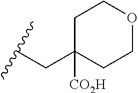 | 319 | 2950 | 0.108 | 55.5 | 118^{a} | 0.28 | — |
| G | 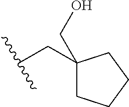 | 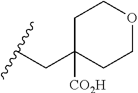 | | | | 33.0 | 79.0^{a} | <0.27 | — |
| H | 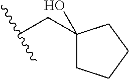 | 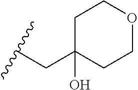 | | | | 30.8 | 104^{a} 50^{b} | 0.83 | — |
| J | 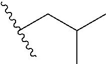 | 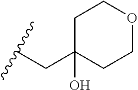 | | | | 39.1 | 55^{b} | 0.78 | 56 |

^{a}96-well format; ^{b}384-well format.

When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations to the invention, the scope of which is defined by the appended claims.

1. (R)-4-((4-((4-(tetrahydrofuran-3-yloxy)benzo[d]isoxazol-3-yloxy)methyl)piperidin-1-yl)methyl)tetrahydro-2H-pyran-4-ol, or a pharmaceutically acceptable salt thereof.

2. A compound of formula X: 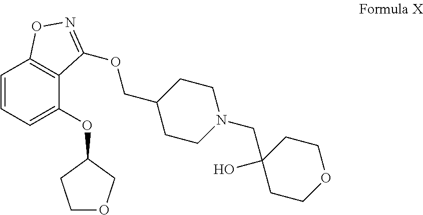
or a pharmaceutically acceptable salt thereof.

3. A pharmaceutical composition comprising the compound of claim 1, or a pharmaceutically acceptable salt thereof, and a pharmaceutically acceptable carrier.

4. A method of treating a neurodegenerative disease or disorder, the method comprising administering a therapeutically effective amount of the compound of claim 1, or a pharmaceutically acceptable salt thereof.

5. The method according to claim 4, wherein the neurodegenerative disease or disorder is dementia, Alzheimer's disease, depression, psychosis, schizophrenia, anxiety, mood disorders, attention deficit/hyperactivity disorder, or attention deficit disorder.

6. The method according to claim 5, wherein the neurodegenerative disease or disorder is Alzheimer's disease.

7. The method according to claim 5, wherein the neurodegenerative disease is dementia.

8. The method of claim 4, wherein the therapeutically effective amount ranges from about 0.01 mg/kg to about 100 mg/kg.
